(19) United States
(12) Patent Application Publication
(10) Pub. No.: US 2008/0200956 A1
(43) Pub. Date: Aug. 21, 2008

(54) Low Profile Orthopedic Fastener Assembly Having Enhanced Flexibility

(75) Inventors: Paul Beckwith, Fishers, IN (US); James Davis, Indianapolis, IN (US)

Correspondence Address: LEVEQUE INTELLECTUAL PROPERTY LAW, P.C., 221 EAST CHURCH STREET, FREDERICK, MD, 21701, US

(73) Assignee: Tutela Medicus, LLC, Indianapolis, IN (US)

(21) Appl. No.: 12/032,040

(22) Filed: Feb. 15, 2008

Related U.S. Application Data
(60) Provisional application No. 60/890,553, filed on Feb. 19, 2007.

(51) Int. Cl. A61B 17/56; A61B 17/08

(52) U.S. Cl. 606/308; 606/301; 606/151

(57) ABSTRACT

An orthopedic fastener assembly for stabilizing and fixing spinal column, bone sections and joints of the body. The orthopedic fastener assembly having a screw-pin anchor, a collet/inner coupling and head/outer coupling. Once assembled, the present invention has an even-lower profile, providing for multiple adjustments and enhanced flexibility, yet is easier to manufacture because of its greater tolerances than previously known assemblies.

### PRIORITY CLAIM

This application claims priority pursuant to 35 U.S.C. § 119(e) to the provisional U.S. patent application filed Feb. 19, 2007 and identified by Application No. 60/890,553, which is hereby incorporated by reference in its entirety.

### BACKGROUND

The mechanical hardware used to immobilize portions of the vertebral/spinal column can generally involve a series of bone screw-pins and metal rods or plates secured to the vertebral bodies of interest. When the vertebra/spine procedure is performed posteriorly, it can be practice to place bone screw-pins into the vertebral bodies and then connect a metal connecting rod between the bone screw-pins thus creating a mostly rigid structure between adjacent vertebral bodies. When the vertebra/spine procedure is performed anteriorly, it can be practice to attach a thin metal plate directly to the vertebral bodies and secure them to each respective vertebral level using one or more bone screw-pins.

Many conventional devices for locking a vertebral/spinal rod to a fixation hook or screw or pins do not offer the needed variability and flexibility to allow the vertebral/spinal rod to be easily connected to adjacent vertebrae, which are not aligned on the same plane. In some cases the use of these devices may be one-time permanently implanted in the subject. In other cases, the devices may be implanted only as a temporary means of stabilizing or fixing the bones or bone fragments, with subsequent removal when no longer needed. It is also common that device implants that were intended to be one-time permanent may require subsequent procedures or adjustments as the skeletal dynamics of the patient condition warrant. For these reasons, it is preferable that an implanted device be provided, which can be locked and unlocked as desired by the practitioner and have as many adjustments as possible.

To meet the problem of securely connecting adjacent vertebrae, not on a common plane, a requirement exists to provide a low profile spinal coupling assembly that can be inserted and adjusted from the vertebral bone as desired and to provide the instrumentation that can facilitate quick locking and unlocking of such a low profile spinal coupling without having to apply any additional torque to the implanted screw-pin anchor settled in the vertebrae.

During the vertebral/spinal fixation, the several bone screw-pins are threaded into the different vertebrae according to the anatomy of each vertebra. This results in a series of screw-pins without uniformity in angle or alignment. To compensate for these anomalies, the connection between the head of the screw-pins and the clamp bodies pivots or swivels to capture the connector rod. In some instances, the rod must be bent because the screw-pins are so far out of line or the intended correction is so severe. In other cases, a link may be used to secure the rod relative to the bone screw-pin. To avoid application of any more torque to the bone screw-pin, the connector rod is secured to the bone screw-pin by a linear motion which applies compressive force through clamp to the rod and the head of the screw-pin. Recent well documented examples of this listed numerically are: US 2008/0027432 A1 Strauss's Multi-Planar Taper Lock Screw; US 2007/0167949 A1 Alaric's Screw Systems and Methods for use in Stabilization of Bone Structures; US 2007/0093817 A1 Barros's Spinal Fixation System Having Locking and Unlocking Devices for use with a Multi-Planar, Taper Lock Screw; US 2005/0096653 A1 Doubler's Bone Fixation System With Low Profile Coupling; U.S. Pat. No. 7,090,674 Doubler's bone fixation system with low profile fastener.

There is a general need for an orthopedic fastener prosthesis and assembly with an even-lower profile, maximized flexibility and adjustments for both practitioner and patients, particularly small stature persons. There is also a need for greater tolerances in manufacturing such a product.

### BRIEF DESCRIPTION OF THE DRAWINGS

The features of the invention believed to be novel are set forth with particularity in the appended claims. The invention itself however, both as to organization and method of operation, together with objects and advantages thereof, may be best understood by reference to the following detailed description of the invention, which describes certain exemplary embodiments of the invention, taken in conjunction with the accompanying drawings in which:

FIG. 1 is a screw-pin anchor, in accordance with the prior art.

FIG. 2 is a collet/inner coupling, in accordance with various embodiments.

FIG. 3 is a side view of a collet/inner coupling, in accordance with various embodiments.

FIG. 4 is another side view of a collet/inner coupling, in accordance with various embodiments.

FIG. 5 is a head/outer coupling, in accordance with various embodiments.

FIG. 6 is a cross-sectional view of a head/outer coupling, in accordance with various embodiments.

FIG. 7 is a to view of a head/outer coupling, in accordance with various embodiments.

FIG. 8 is a side view of a head/outer coupling, in accordance with various embodiments.

FIG. 9 is another side view of a head/outer coupling, in accordance with various embodiments.

FIG. 10 is a cross-sectional view of a secured assembly, without rod, in accordance with various embodiments.

FIG. 11 is a perspective view of a secured assembly, without rod, in accordance with various embodiments.

FIG. 12 is a cross-sectional view of a secured assembly, with rod, in accordance with various embodiments.

FIG. 13 is a perspective view of a secured assembly, with rod, in accordance with the prior art.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

While this invention is susceptible of embodiment in many different forms, there is shown in the drawings and will herein be described in detail specific embodiments, with the understanding that the present disclosure is to be considered as an example of the principles of the invention and not intended to limit the invention to the specific embodiments shown and described. In the description below, like reference numerals are used to describe the same, similar or corresponding parts in the several views of the drawings.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

Reference throughout this document to “one embodiment”, “certain embodiments”, “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

This invention relates to an orthopedic fastener and, in particular, to prosthesis and assemblies for stabilizing and fixing spinal column, bone sections and joints of the body. In accordance with various embodiments, the present invention provides for an even-lower profile orthopedic fastener assembly, such as a polyaxial or subcutaneous orthopedic fastener assembly comprising a screw-pin anchor, a collet/inner coupling and head/outer coupling. Once assembled, the present invention has an even-lower profile, providing for multiple adjustments and enhanced flexibility, yet is easier to manufacture because of its greater tolerances than previously known assemblies.

An orthopedic fastener assembly operable to attach a connecting rod to bone, then, has a bone engaging screw-pin anchor, the screw-pin anchor having an anchoring portion configured as an external helical thread screw-pin shaft for penetrating bone through the application of torque and an upper portion that terminates in a spherical screw-pin head; a collet/inner coupling comprising a top section and a bottom section, said top section having a diameter larger than the bottom section, wherein said top section of said collet/inner coupling further comprises a first u-shaped rod-slot and a plurality of linear-flex rod-grooves located in an internal surface of the collet below a plurality of beveled snap slot-lips at the top section of said collet/inner coupling and wherein said bottom section of said collet/inner coupling comprising a first passageway and a mating spherical pocket located in the bottom face for compressive engagement atop the upper spherical portion of the spherical screw-pin head; and a head/outer coupling comprising a top section, an irregular middle section, and a bottom section, said top section having an internal diameter larger than the bottom section, wherein the internal diameter of said head/outer coupling tapers outwardly at said top section to said irregular middle section and further tapers inwardly from said irregular middle section to said bottom section thereby forming a plurality of multiple interior tapered surfaces for snap fit engagement of the outer surface of said collet/inner coupling and wherein said head/outer coupling further comprises a second passageway located below the bottom section of said collet/inner coupling for gripping of the spherical screw-pin head and a second u-shaped rod-slot configured to mate with the first u-shaped rod-slot to accommodate snap fit engagement of a connecting rod.

In accordance with various embodiments, the screw-pin anchor has a shaft, ribbed head and screw-pin head recess for engagement with drivers.

In accordance with various embodiments, the collet/inner coupling comprises a u-shaped rod-slot; a top section with semi-circular slot lips and rod-grooves; an irregular middle section with an alignment orifice; a lower section having a circumferential undercut spring-ring groove with arch access orifices and a bottom face spherical cup-pocket below a first passageway, such as an axial or subcutaneous passageway.

The various embodiments provide for the head/outer coupling to have a multi-tapered second passageway; a top section having opposed semi-circular lips, opposed u-shaped rod-slots, a guide hole for dimple manufacturing and sidewall arch access orifices; a curved outer wall and a beveled bottom section.

The various embodiments may moreover provide a head/outer coupling comprising a multi-tapered second passageway; a top section having opposed semi-circular lips, opposed u-shaped rod-slots, a guide hole for dimple manufacturing, sidewall arch access orifices and cannulated bone fastener which allows for minimally invasive approach and subcutaneous rod placement, to bones along a guide wire; a curved outer wall and a beveled bottom section.

Well known in the art, the screw-pin anchor 10 as best shown in FIG. 1 includes a screw-pin shaft 11 which defines an external helical thread for penetrating bone through the application of torque. The upper portion of the screw-pin shaft 11 terminates in a screw-pin head 12 that is generally spherical in part and ribbed 13 for gripping purposes. The uppermost flatter surface includes a screw-pin head recess 14, which has a configuration that is complementary to the shape of a tightening and/or loosening tool. The screw-pin head recess 14 may engage a screw-pin driver or more specifically a hex screw driver (not illustrated). The convex screw-pin head recess 14 also enables a close proximity of the connecting or connector rod 15, hence the even-lower profile. Also, the bone into which the screw-pin is driven may be prepared in advance of inserting the screw-pin in any suitable manner within the discretion of the surgeon such as by drilling and optionally tapping a hole to receive the screw-pin. Well known in the art, the screw-pins are capable of joining connecting rods to multiple vertebrae, which are aligned in the vertebral/spinal column on different planes due to the natural curvature of the vertebra/spine.

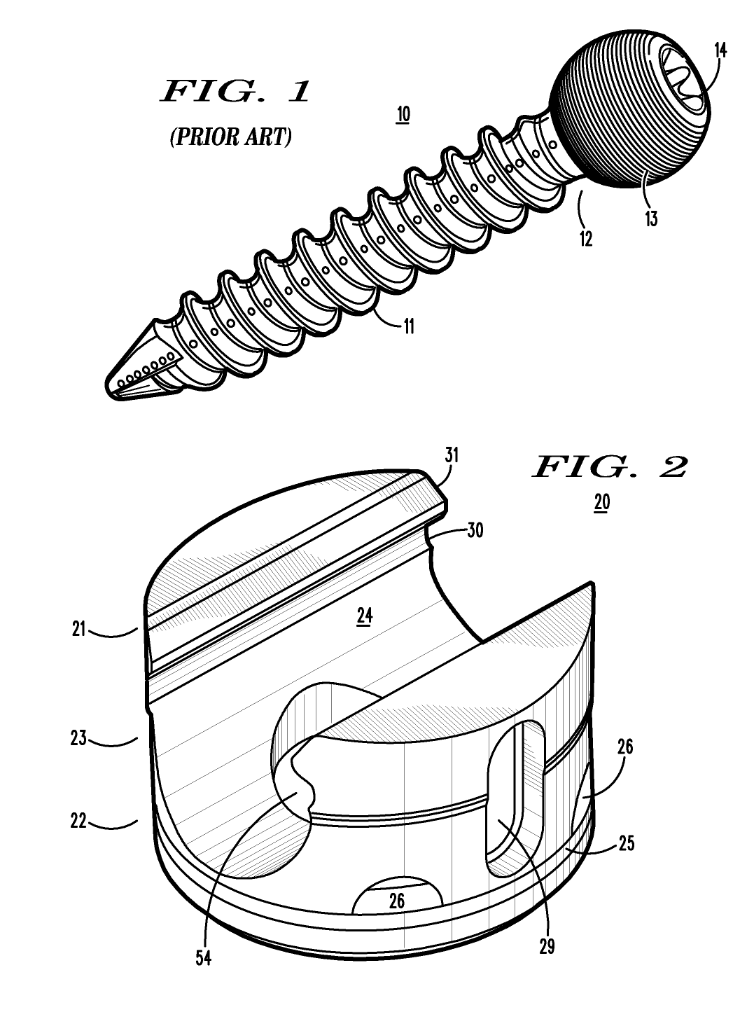

As shown in FIG. 2, the collet/inner coupling 20 defining a top section 21 and a bottom section 22, the top section 21 having a diameter larger than the bottom section 22. The top section 21 further includes a partially cylindrical u-shaped rod-slot 24 for snap fit engagement of the connecting rod 15 component and additionally, linear-flex rod-grooves 30 located below the beveled snap slot-lips 31 for flexible compressive adjustment of connecting rod 15 in rod-slot 24. As shown in FIG. 2, the bottom section 22 includes an external circumferential undercut spring-ring groove 25 including arch access orifices 26 extending upwardly from the circumferential undercut spring-ring groove 25. The novel spring-ring groove 25 and arched access orifices 26 are for both flexible compressive adjustment atop the screw-pin head 12 and axial flexibility of the axial passageway 54. The multiple arch access orifices 26 are also used for engagement of a manipulation device (not shown). Also shown in FIG. 2 is an alignment orifice 29 in one side of the irregular middle section 23 for upward and downward manipulation with a dimple 63 formed to prevent rotation of the cylindrical collet/inner coupling 20 with respect to the cylindrical head/outer coupling 50.

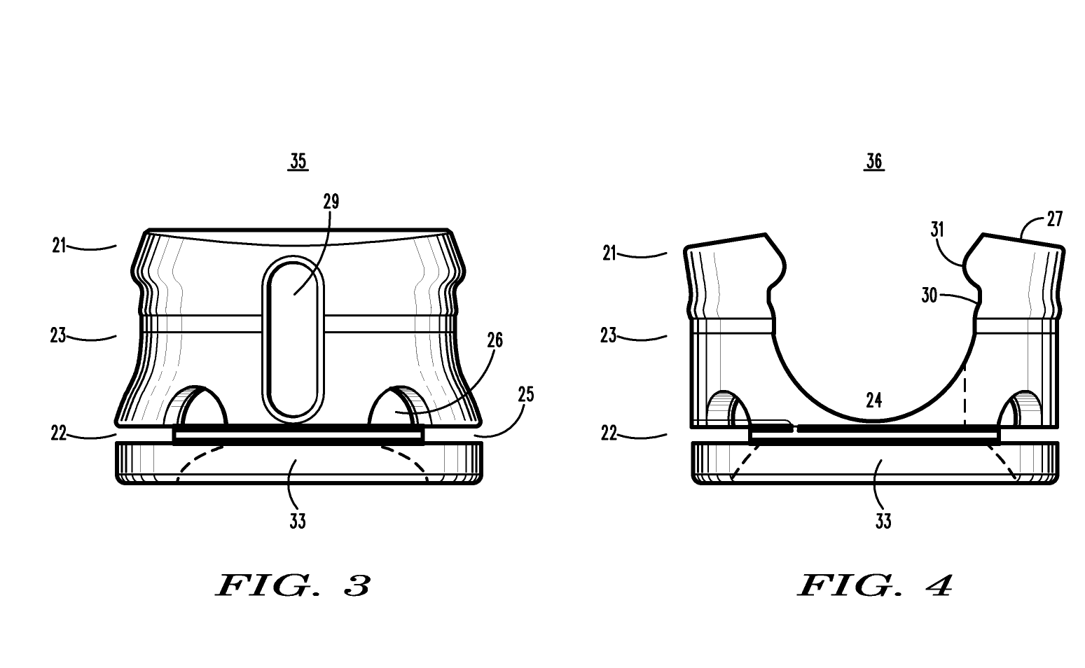

In assembly the cylindrical collet/inner coupling 20 sits flexibly adjustable atop the screw-pin head 12 and flexibly adjustable inside the cylindrical head/outer coupling 50. As shown in FIG. 3, cylindrical sidewall 35 side view includes a flared top section 21 and flared beveled bottom section 22 interconnected by an irregular middle section 23. The flared top section 21 is for slightly flexible compression around the connecting rod 15 as well as the snap fitting into and out of the cylindrical head/outer coupling 50. The larger flared beveled bottom section 22 includes an external circumferential undercut spring-ring groove 25, including arch access orifices 26 extending upwardly from the circumferential undercut spring-ring groove 25. In the bottom face (not shown) is a springy spherical cup-pocket 33 shaped to match and fit on top of the screw-pin head 12. Further, this side includes the alignment orifice 29 for the dimple 63.

As shown in FIG. 4, another cylindrical sidewall 36 side view includes a top section 21 and beveled bottom section 22 interconnected by an irregular middle section 23. The top section 21 includes a slightly higher top face 27 leading into the curved beveled rod-slot lip 31 above the linear-flex rod-groove 30 above the partially circumferential u-shaped rod-slot 24. The beveled bottom section 22 includes an external circumferential undercut spring-ring groove 25, spaced slightly above the bottom section 22 and includes arch access orifices 26 extending upwardly from the circumferential undercut spring-ring groove 25. In the bottom face (not shown) is a springy spherical cup-pocket 33 shaped to match and fit on top of the screw-pin anchor 10.

In assembly and application, the cylindrical collet/inner couplings subtle design enable polyaxial positioning having free-spaces, compressions, spring, bounce, movements, flexibility, adjustments, for a superior performance. It also permits greater tolerances in manufacturing than existing similar products on the market.

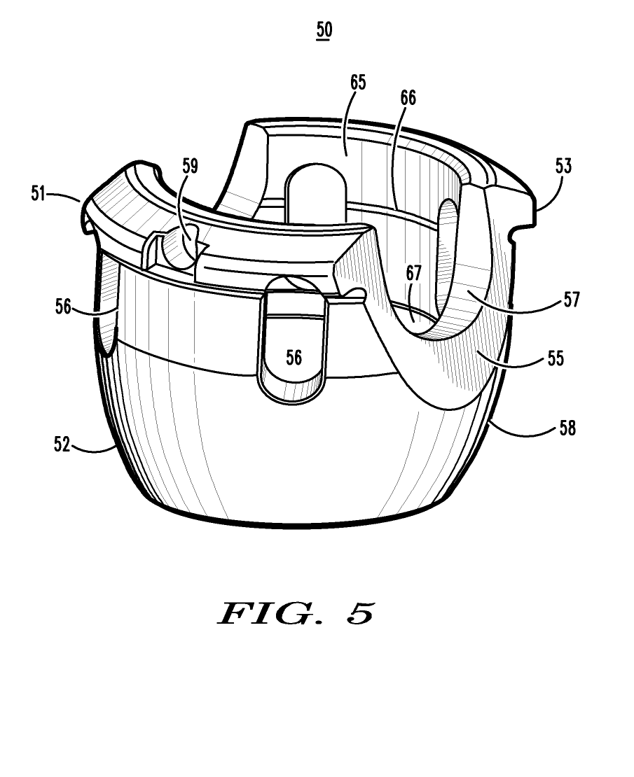

As shown in FIG. 5, the cylindrical head/outer coupling 50 defining opposed top sections 51 and a beveled curved bottom section 52. The top section 51 includes opposed beveled semi-circular lips 53 on both sides but only one side includes a guide-hole 59. On both sides are multiple arch access orifices 56 going completely through the wall of top section 51. The top section 51 further includes directly opposed partially cylindrical u-shaped rod-slot edges 57 and tapered outer side faces 55. The bottom section 52 includes a cylindrical outer curved wall 58. Both top section 51 and bottom section 52 encircle an axial passageway 54 (not shown) inner wall 65, with an upper inner tapered flange 66 and a lower inner tapered flange 67.

To assemble, the screw-pin anchor 10 simply fits through the axial passageway 54 catching the grip of the cylindrical head/outer coupling 50, followed further by the cylindrical collet/inner coupling 20 snap fitting inside. To disassemble a tool is requires to snap upwards on the arch access orifices 26.

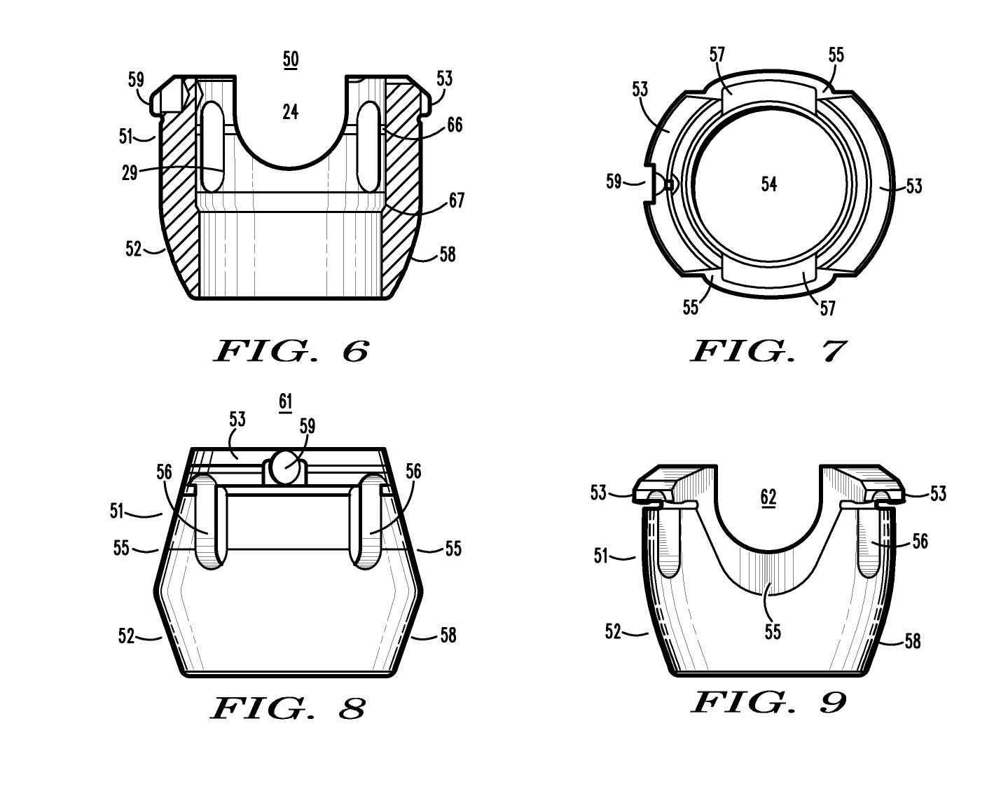

As shown in FIG. 6, the cylindrical head/outer coupling 50 cross section view includes a top section 51 having opposed semi-circular lips 53 with one side having a guide-hole 59. The top section 51 further includes multiple arch access orifices 56 and a partially cylindrical u-shaped rod-slot 24. The curved beveled bottom section 52 includes an outer curved wall 58 of the cylindrical head/outer coupling 50. The cylindrical head/outer coupling 50 interior side wall 65 includes an upper outwardly tapered flange 66, a lower inwardly tapered flange 67 and a punched dimple 63.

As shown in FIG. 7, top view of the cylindrical head/outer coupling 50 including the axial passageway 54, the opposed semi-circular lips 53 with one side having a guide-hole 59, the directly opposed rod-slot edges 57 and opposed tapered outer side faces 55.

As shown in FIG. 8, a cylindrical sidewall 61 side view includes a top section 51 having a semi-circular lip with the guide hole 59, opposed tapered outer side faces 55 and multiple arch access orifices 56. The beveled bottom section 52 includes the cylindrical outer curved wall 58.

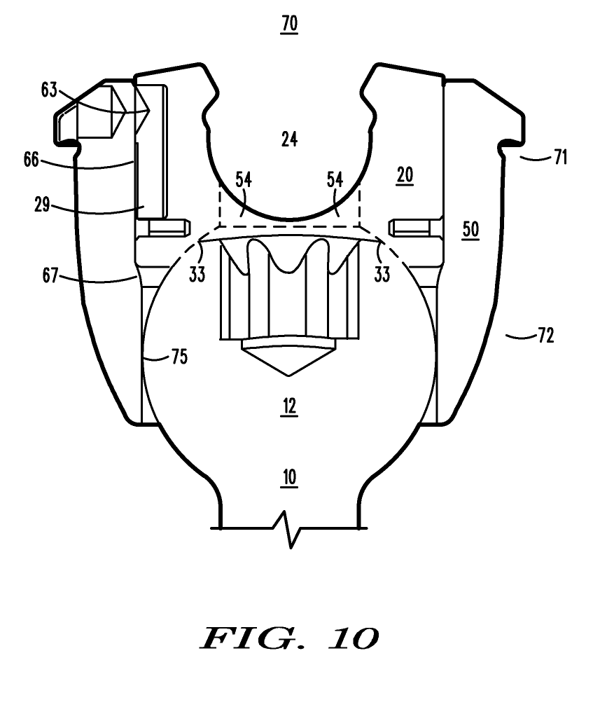

As shown in FIG. 9, cylindrical sidewall 62 side view includes beveled bottom section 52 with the cylindrical outer curved wall 58. The top section 51 includes opposed outer semi-circular lips 53, multiple arch access orifices 56, a partially cylindrical u-shaped rod-slot edge 57 and a tapered outer side face 55.

The combination of the specially designed couplings on each of and between the cylindrical collet/inner coupling 20, the cylindrical head/outer coupling 50, atop the well known screw-pin anchor 10, allow for an ease of assembly and application. Additionally it is more productive and efficient to manufacture than current similar products because of greater tolerances. Further, all material types well known in the art may be used in manufacturing.

As shown in FIG. 10, the cross section of secured assembly 70 includes an assembled top section 71 and assembled bottom section 72 of the screw-pin anchor 10, the cylindrical collet/inner coupling 20 and cylindrical head/outer coupling 50. The assembled top section 71 includes both cylindrical collet/inner coupling 20 interconnected with cylindrical head/outer coupling 50 in a snap fit engagement which can be disengaged with appropriate tools and force. Also included is the empty partially cylindrical u-shaped rod-slot 24 sitting above the axial passageway 54, in flexible bouncing axial contact with the springy spherical cup-pocket 33, further in flexible bouncing axial contact with the screw-pin head 12. The assembled top section also shows the guide-hole dimple 63 extending into the alignment orifice 29, in order to prevent rotation of the cylindrical collet/inner coupling 20 with respect to the cylindrical head/outer coupling 50. This dimple 63 extending into the alignment orifice 29 is not precise and can be manipulated by the up or down snap force movement of the collet/inner coupling 20. Further not in contact but in close proximity, is the upper inner tapered flange 66 compressing inwardly on the top section 21. Further, the lower inner tapered flange 67 compressing inwardly on the lower section 22. Further, the circumferential undercut spring-ring groove 25 and screw-pin head free-spaces 76 & 77. Additionally when assembled, the bottom section 72 of the cylindrical head/outer coupling firmly grips the ribbed 13 screw-pin head 12 anywhere along the contact plane 75 for the lowest possible profile.

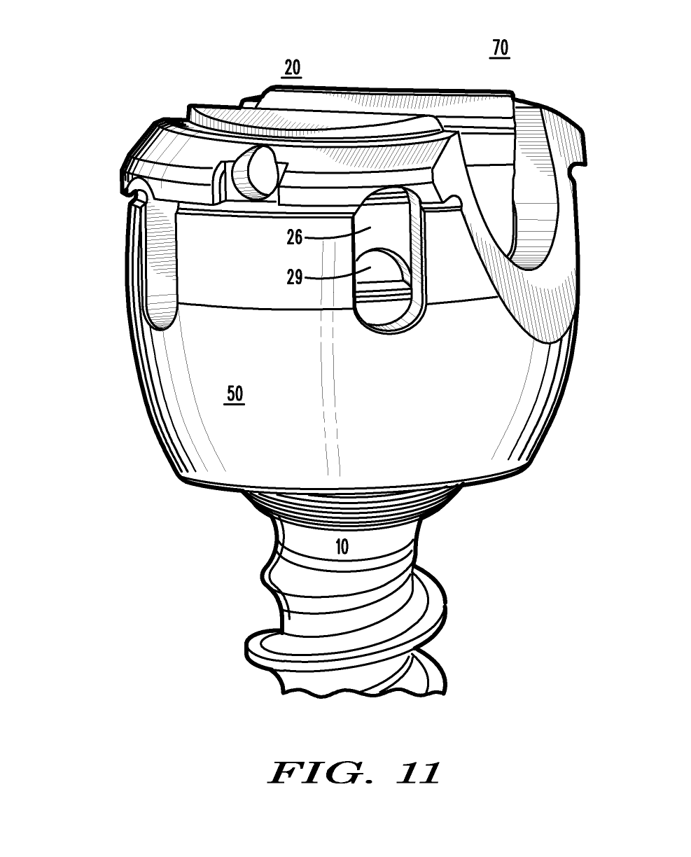

As shown in FIG. 11 secured assembly 70 without rod perspective shows interconnected screw-pin anchor 10 with cylindrical collet/inner coupling 20 with cylindrical head/outer coupling 50. Further included is the alignment orifice 29 alignment with the multiple arch access orifices 26.

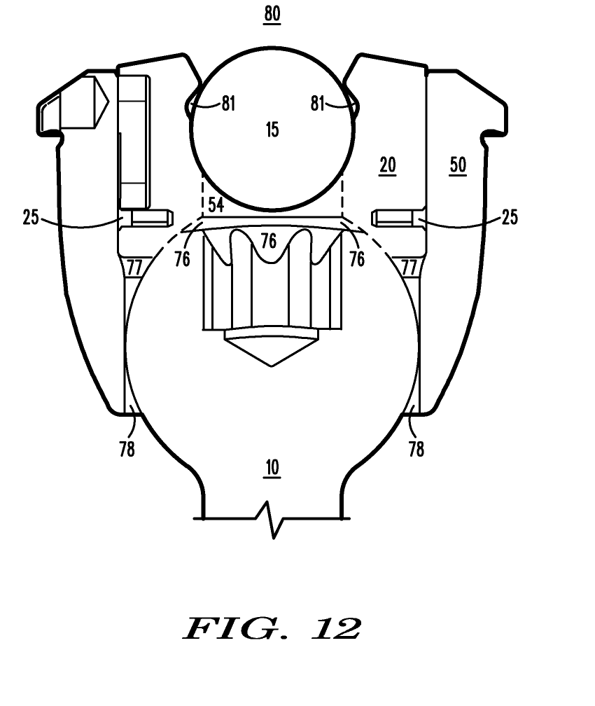

As shown in FIG. 12 secured assembly 80 with connecting rod 15 cross section shows the tolerances of circumferential screw-pin head free-spaces 76, 77 and 78, connecting rod linear free-spaces 81 and circumferential spring-ring groove 25 free-space in relation to the secured assembled screw-pin anchor 10 with cylindrical collet/inner coupling 20 with cylindrical head/outer coupling 50 with connecting rod 15.

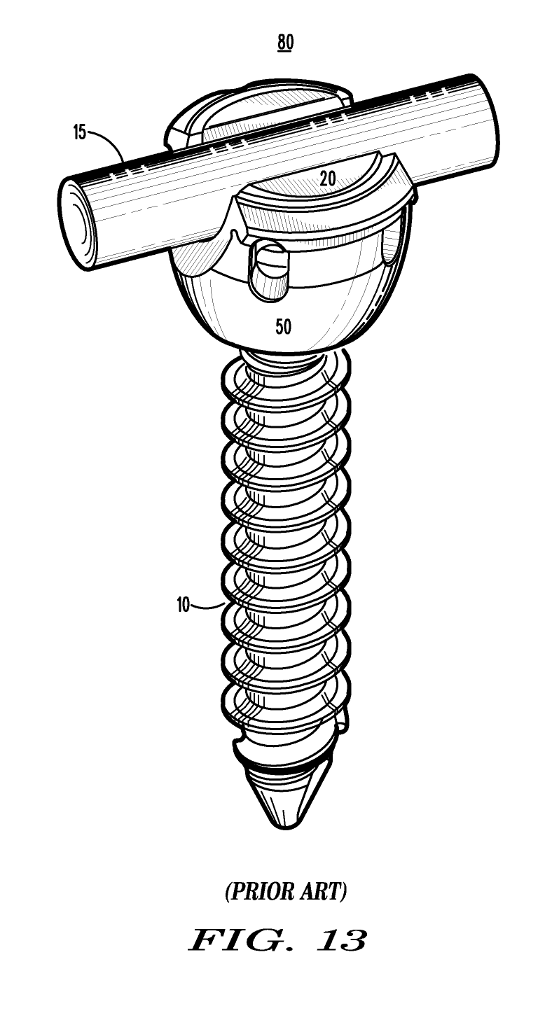

As shown in FIG. 13, the secured assembly 80 with connecting rod 15 perspective shows securely assembled screw-pin anchor 10 with cylindrical collet/inner coupling 20 with cylindrical head/outer coupling 50 with connected rod 15.

It is understood that collet/inner coupling 20 and head/outer coupling 50, and rod-slot(s) 24, while described as cylindrical, partially cylindrical, u-shaped or the like herein, are not so limited in shape. They may encompass a variety of shapes, including cylindrical, oval, u-shaped, substantially cylindrical, or other chosen shapes without departing from the spirit and scope of the current invention.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. An orthopedic fastener assembly operable to attach a connecting rod to bone, comprising:
a bone engaging screw-pin anchor, the screw-pin anchor having an anchoring portion configured as an external helical thread screw-pin shaft for penetrating bone through the application of torque and an upper portion that terminates in a spherical screw-pin head;
a collet/inner coupling comprising a top section and a bottom section, said top section having a diameter larger than the bottom section,
wherein said top section of said collet/inner coupling further comprises a first u-shaped rod-slot and a plurality of linear-flex rod-grooves located in an internal surface of the collet below a plurality of beveled snap slot-lips at the top section of said collet/inner coupling;
said bottom section of said collet/inner coupling comprising a first passageway and a mating spherical pocket located in the bottom face for compressive engagement atop the upper spherical portion of the spherical screw-pin head; and
a head/outer coupling comprising a top section, an irregular middle section, and a bottom section, said top section having an internal diameter larger than the bottom section,
wherein the internal diameter of said head/outer coupling tapers outwardly at said top section to said irregular middle section and further tapers inwardly from said irregular middle section to said bottom section thereby forming a plurality of multiple interior tapered surfaces for snap fit engagement of the outer surface of said collet/inner coupling,
wherein said head/outer coupling further comprises a second passageway located below the bottom section of said collet/inner coupling for gripping of the spherical screw-pin head and a second u-shaped rod-slot configured to mate with the first u-shaped rod-slot to accommodate snap fit engagement of a connecting rod.

2. The assembly according to claim 1, wherein said bottom section of said collet/inner coupling further comprises a circumferential undercut groove with a plurality of arched access orifices extending upwardly from said circumferential undercut groove.

3. The assembly according to claim 2, wherein said circumferential undercut groove with the plurality of arched access orifices allows flexible compressive adjustment of said collet atop the spherical screw-pin head.

4. The assembly according to claim 2, wherein the circumferential undercut groove is an external circumferential undercut spring-ring groove.

5. The assembly according to claim 2, wherein the plurality of arch access orifices located in the circumferential undercut groove of said collet/inner coupling are for further engagement of an extraction device.

6. The assembly according to claim 2, wherein said head/outer coupling further comprises multiple slots to allow an extraction instrument to access the plurality of arch orifices in the circumferential undercut groove located in the bottom section of the collet/inner coupling.

7. The assembly according to claim 1, wherein the plurality of linear-flex rod-grooves located in the internal surface at the top section of said collet/inner coupling allows for partial compression of the first partially circumferential u-shaped rod-slot for engagement of a connecting rod.

8. The assembly according to claim 1, wherein: said linear-flex rod-grooves located in the inner surface of said collet allow for flexible compressive adjustment of a connecting rod in said first u-shaped rod-slot.

9. The assembly according to claim 1, wherein: the tapered inner surfaces at the top section of said head/outer coupling component compress the collet/inner coupling to a connecting rod.

10. The assembly according to claim 1, wherein said head/outer coupling further comprises a plurality of opposed beveled semi-circular lips on the top section of said head/outer coupling;

11. The assembly according to claim 10, wherein said head/outer coupling further comprises a guide-hole located in a semi-circular lip of said opposed beveled semi-circular lips for punching out a dimple into an alignment orifice for prevention of rotation between said collet/inner coupling and said head/outer coupling, said dimple being manipulated by an up and down snap force.

12. The assembly according to claim 1, wherein the assembly is assembled by placing the screw-pin anchor through the second passageway of the head/outer coupling and snap fitting or extracting said collet/inner coupling inside said head/outer coupling.

13. The assembly according to claim 1, wherein the assembly further comprises a connecting rod configured for placement adjacent and along a length of spinal columns or bone sections.

14. The assembly according to claim 1, wherein the first and second passageways are subcutaneous passageways.

15. The assembly according to claim 1, wherein the first and passageways of said bottom section of said collet/inner coupling are axial passageways.

16. The assembly according to claim 1, wherein the second passageway of said head/outer coupling further provides for adjustment and detachment of the spherical screw-pin head.

17. The assembly according to claim 1, wherein the screw-pin anchor is a cannulated bone engaging screw-pin anchor.

18. The assembly according to claim 1, wherein said collet/inner coupling and said head/outer coupling are substantially cylindrical and said first and second u-shaped rod-slots are partially cylindrical.
